(19) United States
(12) Patent Application Publication
(10) Pub. No.: US 2002/0048754 A1
(43) Pub. Date: Apr. 25, 2002

(54) Apparatus and method for processing multiple arrays of biological probes

(76) Inventors: David J. Lockhart, Del Mar, CA (US); Patrick P. Zarrinkar, San Diego, CA (US); James K. Mainquist, San Diego, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(21) Appl. No.: 09/682,838

(22) Filed: Oct. 23, 2001

Related U.S. Application Data
(60) Provisional application No. 60/242,859, filed on Oct. 24, 2000.
(60) Provisional application No. 60/244,817, filed on Oct. 31, 2000.

(51) Int. Cl. C12Q 1/68; C12M 1/34

(52) U.S. Cl. 435/6; 435/287.2

(57) ABSTRACT

Apparatuses and methods are described that enable high-throughput processing (e.g., hybridizing, washing, and staining) of microarrays. This high-throughput processing is achieved in part by combining the capabilities for separate hybridization of multiple arrays in fluidically separated hybridization chambers with parallel processing of those arrays in a single fluidic chamber during certain processing stages. In some implementations, the apparatus includes a separating member constructed and arranged so that, when it is disposed in a first position the microarrays are fluidically separated from each other. When the separating member is removed, the microarrays are fluidically coupled with each other. Thus, separate microarray hybridization chambers may readily be converted to a single fluidic chamber by moving the separating member.

### CROSS REFERENCE TO RELATED APPLICATIONS

The present application claims priority from U.S. Provisional Patent Application Serial No. 60/242,859, filed Oct. 24, 2000, which is hereby incorporated by reference herein in its entirety for all purposes; and from U.S. Provisional Patent Application Serial No. 60/244,817, filed Oct. 31, 2000.

### BACKGROUND OF INVENTION

1. Field of the Invention

The present invention relates to apparatuses for microarray processing and, in particular, to assemblies that hold, process, or transport arrays of biological probes.

2. Related Art

Microarrays with extremely large number of probes are manufactured by methods described in U.S. Pat. Nos. 5,143,854; 5,384,261; 5,405,783; 5,412,087; 5,424,186; 5,445,934; 5,744,305; 5,800,992; 6,040,138; 6,040,193; 6,140,044; 6,150,147; 6,153,743; and 6,291,183; and in PCT Application PCT/US91/08693, published as WO 92/10092; all of which are hereby incorporated herein in their entireties by reference. The probes may have dimensions from about 10 microns to 100 microns. Each probe may include several million DNA molecules. After exposing the microarray to target molecules under selected test conditions, scanning devices can examine locations in the array to determine whether target molecules have interacted with probes at those locations.

Microarray technology has been used to analyze a large number of complex biochemical reactions and systems. This technology provides a massively parallel form of analysis that increases data collection per unit time, decreases the overall time required for analysis, and uses smaller sample volumes and reagent volumes. For these and other reasons, microarray technology is well suited for genomic research. For example, microarrays have been used for monitoring the expression levels of a multiplicity of genes. See, e.g., U.S. Pat. Nos. 6,040,138; 6,027,880; and 6,185,561; all of which are hereby incorporated in their entireties herein by reference for all purposes.

### SUMMARY OF INVENTION

Apparatuses and methods are described herein that enable high-throughput processing (e.g., hybridizing, washing, and staining) of microarrays. Methods previously have been described for processing multiple microarray assays in U.S. Pat. Nos. 5,545,531 and 5,874,219, both of which are hereby incorporated herein by reference in their entireties for all purposes. Those previously described multiple-processing methods provide significant advantages over traditional approaches in which microarrays are separately processed. Various apparatuses and methods of the present invention provide further advantages by, among other things, combining the capabilities for separate hybridization of multiple arrays in fluidically separated hybridization chambers with parallel processing of those arrays in a single fluidic chamber during certain processing stages.

More specifically, in accordance with one embodiment a method is described for analyzing nucleic acids using a plurality of nucleic acid microarrays. The method includes the steps of: (1) preparing a cell sample having nucleic acids; and (2) contacting the sample with an apparatus that comprises one or more containing members constructed and arranged to contain the plurality of nucleic acid microarrays, and a separating member constructed and arranged so that, when the separating member is disposed in a first position with respect to the containing members, at least two of the plurality of nucleic acid microarrays are fluidically separated from each other by the separating member, and when the separating member is disposed in a second position with respect to the containing members, the at least two microarrays are fluidically coupled with each other.

In accordance with another embodiment, an apparatus is described for processing a plurality of microarrays disposed on a substrate. The apparatus includes one or more containing members that contain the substrate. The apparatus also includes a separating member constructed and arranged so that, when the separating member is disposed in a first position with respect to the containing members, at least two of the plurality of microarrays are fluidically separated from each other by the separating member.

Moreover, when the separating member is disposed in a second position with respect to the containing members, the at least two microarrays are fluidically coupled with each other. Thus, in some implementations, separate microarray hybridization chambers are converted to a single fluidic chamber by moving the separating member.

In these contexts, the term fluidically separated and grammatical variants are used broadly to mean, for example, that a fluid disposed so as to interact with one microarray does not interact with another microarray. Similarly, the term fluidically coupled and grammatical variants are used broadly to mean, for example, that a fluid may interact with more than one microarray by, for instance, flowing over both microarrays in the same fluidic cell or chamber. The fluid may thus be referred to herein as operating in parallel with the two or more microarrays, or being involved in parallel fluidic processes, even though it is not necessary in all implementations that the fluid contact each of the microarrays evenly, or at the same instant.

In some of these embodiments, the one or more containing members include a first segment and a second segment in contact with the first segment, wherein the substrate is disposed between the first and second segments. The separating member may be disposed between the first and second segments when the separating member is in the first position, and may be disposed apart from the first and second segments when the separating member is in the second position. The substrate may be retained in place by the first and second segments. The first segment may include a central frame, which may have an inlet port for receiving fluids and an outlet port for expelling fluids. The separating member may include one or more walls constructed and arranged to fluidically separate the at least two microarrays when the separating member is disposed in the first position.

In some implementations, the separating member may include a grid plate that has grid elements determined by walls. For example, a grid element may be a chamber made up of a group of the walls and having one surface made up of a portion of the grid plate lying within the group of walls. In these implementations, each of the at least two microarrays is fluidically separated from each of the other at least two microarrays by a grid element (e.g., grid chamber) when the separating member is disposed in the first position. Each of the at least two microarrays is fluidically coupled with the other at least two microarrays when the separating member is disposed in the second position.

The microarrays may be synthesized probe arrays, wherein the probes comprise oligonucleotides. In some implementations, the oligonucleotides may have been synthesized to the microarrays based, at least in part, on photolithographic methods such as described, for example, in U.S. Pat. No. 5,143,854, incorporated by reference above. In particular, the microarrays may be disposed on a contiguous surface of the substrate comprising what is referred to for convenience herein as a photolithographic wafer. This term refers in this context to wafers of multiple arrays produced, at least in part, by photolithographic processes. The production of microarrays on wafers is described, for example, in U.S. patent application Ser. No. 09/824,931, filed Apr. 3, 2001, which is hereby incorporated by reference herein in its entirety for all purposes. In other implementations, however, wafers or other contiguous substrates may be employed on which microarrays have been synthesized or deposited using any of a variety of known techniques, many of which do not include photolithographic processes. The production of microarrays on wafers is described, for example, in U.S. patent application Ser. No. 09/824,931, filed Apr. 3, 2001, which is hereby incorporated by reference herein in its entirety for all purposes. In other implementations, however, wafers or other contiguous substrates may be employed on which microarrays have been synthesized or deposited using any of a variety of known techniques (such as electrical, mechanical, ink jet, or the like), many of which do not include photolithographic processes.

In accordance with other embodiments, an apparatus is described for processing a plurality of microarrays disposed on a substrate. The apparatus includes one or more containing members including a first segment and a second segment, wherein the substrate is disposed between the first and second segments. The apparatus also includes a separating member including a grid plate having a plurality of grid elements constructed and arranged so that, when the separating member is disposed in a first position with respect to the containing members, at least two of the plurality of microarrays are fluidically separated from each other by one or more of the grid elements, and, when the separating member is disposed in a second position with respect to the containing members, the at least two microarrays are fluidically coupled with each other.

In accordance with further embodiments, a method is described for processing a plurality of microarrays. The method includes the steps of: (1) providing a substrate upon which the microarrays are disposed; (2) fluidically separating at least two of the plurality of microarrays from each other; (3) contacting the at least two microarrays with one or more target solutions while the at least two microarrays are fluidically separated; (4) retaining the fluidic separation of the at least two microarrays for a first period of time sufficient for hybridization reactions, if any, to occur between the target solutions and the at least two microarrays; (5) fluidically coupling the at least two microarrays after the first period has elapsed; and (6) performing one or more parallel fluidic processes on the at least two microarrays based, at least in part, on the fluidic coupling. In some implementations, the method also includes (7) removing at least a portion of the one or more target solutions after the first period has elapsed and prior to performing step (5). The one or more fluidic processes may include removing at least a portion of the one or more target solutions, washing, staining, preserving, or other processes.

In accordance with yet another embodiment, a microarray processing system is described that includes a first segment; a second segment in contact with the first segment; and a processing array positioned between the first segment and the second segment and retained in place by the first and second segments. The processing array may include a plate member between the first and second segment, wherein the plate member includes a first surface; a grid segment containing an array of chamber walls; and a bottom support segment. The grid segment is disposed in a first position between the bottom support segment and the plate member. Multiple processing chambers are thereby formed that each include as a first chamber surface a portion of the first surface of the plate member, as a second chamber surface opposed to the first chamber surface a portion of the bottom support segment, and as walls an array element of the array of chamber walls.

The above embodiments, implementations, and aspects are not necessarily inclusive or exclusive of each other and may be combined in any manner that is non-conflicting and otherwise possible, whether they be presented in association with a same, or a different, aspect of the invention. The description of one embodiment or implementation is not intended to be limiting with respect to other embodiments or implementations. Also, any one or more function, step, operation, or technique described elsewhere in this specification may, in alternative embodiments or implementations, be combined with any one or more function, step, operation, or technique described in the summary. Thus, the above embodiments and implementations are illustrative rather than limiting.

### BRIEF DESCRIPTION OF DRAWINGS

The above and further features will be more clearly appreciated from the following detailed description when taken in conjunction with the accompanying drawings. In the drawings, like reference numerals indicate like structures.

FIG. 1 is a diagram of stacked segments of a processing array assembly in accordance with the present invention;

FIG. 2 is a front view of a grid plate of a processing array assembly in accordance with the present invention;

FIG. 3 is side view of a grid plate of a processing array assembly in accordance with the present invention;

FIG. 4 is a cross section of the grid plate shown in FIG. 2 taken along line A-A;

FIG. 5 is a bottom view of a base plate of a processing array assembly in accordance with the present invention;

FIG. 6 is a top view of a base plate of a processing array assembly in accordance with the present invention;

FIG. 7 is a side view of a base plate of a processing array assembly in accordance with the present invention;

FIG. 8 is a cross section view of region A of the grid plate as shown in FIG. 7;

FIG. 9 is a top view of a top plate of a processing array assembly in accordance with the present invention;

FIG. 10 is a cross section of the top plate shown in FIG. 9 taken along line A-A;

FIG. 11 is a side view of the top plate shown in FIG. 9 taken along line B-B″;

FIG. 12 is a top view of a top support plate of a processing array assembly in accordance with the present invention;

FIG. 13 is a side view of the top support plate shown in FIG. 12 taken along line at A-A″;

FIG. 14 is a top view of a bottom support plate and grid seal of a processing array assembly in accordance with the present invention;

FIG. 15 is a cross section of the bottom support plate and grid seal of in FIG. 14 taken along line A-A″;

FIG. 16 is a side view of the bottom support plate and grid seal in accordance with the present invention;

FIG. 17 is an enlargement of region C of the bottom support plate and grid seal shown in FIG. 14;

FIG. 18 is an enlargement of region B of the cross section of the bottom support plate and grid seal shown in FIG. 15;

FIG. 19 is a top perspective view of an assembled processing array assembly in accordance with the present invention;

FIG. 20 is a cross section view of the assembled processing array assembly in accordance with the present invention;

FIG. 21 is an enlargement of region A of the apparatus shown in FIG. 20;

FIG. 22 is a top view of a view plate of a processing array assembly in accordance with the present invention;

FIG. 23 is a cross section view of the view plate shown in FIG. 22 taken along line A-A;

FIG. 24 is an enlargement of region A of view plate as shown in FIG. 23;

FIG. 25 is a side view of a view plate of a processing array assembly in accordance with the present invention; and

FIG. 26 is a cross sectional view of an edge detail of a processing array assembly in accordance with the present invention.

### DETAILED DESCRIPTION

Apparatuses and methods to process multiple microarrays are described herein with respect to illustrative, non-limiting, implementations. In accordance with some of these illustrated embodiments, a multi-segmented processing array assembly is described. The assembly includes a number of stacked segments allowing for access to different segments within the stack, as desired during use. In one implementation, the multi-segmented processing apparatus includes various segments that, for the sake of convenience only, are called top segment, grid segment, sample segment, base segment and view segment. There may also be seals arranged between two or more of the stacked segments as needed. It should be appreciated that the names of the segments are chosen for convenience, and the assembly may be oriented, for example, with the “top” segment on the lower side, or the “base” segment oriented on an upper side, or the assembly disposed in a generally vertical as opposed to horizontal orientation. Accordingly, “top” is used in this description to characterize an orientation that is on the opposite side of a “base” or “bottom” and should not be taken as a characterization of upper versus lower orientations.

In one embodiment, a grid segment is constructed of an array or grid of generally impermeable walls, forming the four edges of an individual sample chamber. The term generally impermeable is used broadly in this context to mean that chambers are fluidically separated from each other. A grid seal is connected to the grid segment and is designed to uniformly and efficiently seal with very low contact pressure. The grid segment, with its accompanying grid seal is mounted to the base segment. The base segment includes a base plate and a bottom support plate. The grid segment is mounted on top of the base plate. An O-ring is also positioned in the base plate. A sample segment such as a glass plate is positioned atop the grid seal, i.e. on the opposite side of the grid seal from the grid segment. In such an arrangement, the sandwich of the glass plate, grid seal and grid plate creates an array of liquid-tight (i.e., fluidically separated) chambers. Preferably, the seal does not contact any of the microarrays on a sample segment, thus allowing for precise alignment of the glass plate and the grid seal.

Advantageously, this increased precision in aligning the glass plate with the grid seal enables more samples to be positioned on each plate. As an example, conventional automated systems may position specimen plates using the edge of the plate.

In one embodiment, the top segment is comprised of a top support plate and a top plate. The outer surface of the top segment is continuous, stepless, and smooth with a minimal gap between the top support plate and the top plate. It is preferred that such a generally smooth surface be used to reduce the likelihood of contamination, although it should be appreciated that other surface provides can be used, such as undulating, curved, etc. The top segment is positioned on one side (i.e. a “top” side) of the glass plate. On the other side of the glass plate is arranged, preferably in this order, the grid plate, the o-ring, and the base segment. All these segments can be fastened together such that metal to metal contact is achieved between the top segment and the base segment. Thus the outer dimensions of the glass plate and grid plate preferably are selected to be smaller than those of the top and bottom segments so as to allow them to fit within the perimeters of the top and bottom segments. By arranging the top segment and base segment to contact one another allows for seal loading independent of fastener torque such that seal loading is consistent and independent of any fastener variation or fastener torque. Accordingly, operator inconsistency in how tight segments are fastened with one another can be reduced or eliminated.

As noted above, the assembled multi-segmented array may be oriented in any desired direction. The assembly optionally may be turned over by an operator, so the glass plate forms the bottom of individual sample chamber(s) and the grid walls of the grid plate forms the walls between individual sample chambers. A unique sample (e.g., target solution) may be placed in each individual sample chamber within the array. Once sealed, the grid creates a liquid tight seal between each individual chamber within the array. Advantageously, the sealed array may undergo multiple process steps, such as hybridization, shaking, or incubation, for the duration of the reaction.

It is an advantage of some embodiments that the multi-segmented array assembly enables imaging and detecting within the assembly itself. Pre-detection processing may also occur within the assembly. In operation, the bottom support optionally may be removed and a view plate installed, thus creating a flow cell chamber in which predetection processing may occur directly on the samples on the glass plate.

It is a further advantage of some embodiments that manipulations are performed without the need to transfer to an entirely different processing device. Costs for processing reagents, test samples and cleanup may be reduced. Moreover, eliminating the need to transfer samples to a separate device between detection pre-processing steps and the detecting step enables smaller, low volume amounts of samples to be used. As another added advantage in some implementations, because the samples are not removed from the original apparatus, multiple detecting steps may be practiced on a single array by pre-processing for another detection step and moving the apparatus to another detecting device.

In one aspect, the present invention includes a microarray processing system including segmented processing array comprising a plurality of segments arranged in stacked relation, the segmented processing array comprising a first segment, a second segment in contact with the first segment, and a processing array positioned between the first segment and the second segment, and retained in place by the first and second segments. In another aspect of the invention, the microarray processing system includes multiple processing chambers, the processing chambers comprising: a plate member between the first and second segment, a grid segment containing an array of chamber walls, and a bottom support segment, the grid segment positioned between the bottom support segment and the plate member forming a first surface of the processing chambers and the plate member forming a second surface of the processing chambers.

In various embodiments, the processing system is used to process arrays of microarrays on a substrate, such as, for example, a wafer. One type of microarray produced in groups on a wafer and then, conventionally, diced to allow individual packaging is the Affymetrix® GeneChip® probe array available from Affymetrix, Inc. of Santa Clara, Calif. The GeneChip® probe array is synthesized using photolithographic methods, as noted above. Various other techniques for synthesizing microarrays (i.e., producing probes in situ) are available. While illustrated implementations of the present invention may be described herein with respect to synthesized microarrays, and the GeneChip® type array in particular, it should be noted that the apparatuses and methods described herein may be applied with respect to many other types of probe arrays and, more generally, with respect to numerous parallel biological assays produced in accordance with other conventional technologies and/or produced in accordance with techniques that may be developed in the future. For example, aspects of the apparatuses and methods described herein may, in some implementations, be applied to parallel assays of nucleic acids, PCR products generated from cDNA clones, proteins, antibodies, or many other biological materials. These materials may be disposed on slides, on substrates employed for GeneChip® arrays, or on beads, optical fibers, or other substrates, supports, or media (all or any of which may hereafter generally and collectively be referred to as substrates). See also, U.S. Pat. No. 5,143,854 for additional substrates. Moreover, with respect to some implementations in which the context so indicates or allows, the probes need not be immobilized in or on a substrate, and, if immobilized, need not be disposed in regular patterns or arrays. For convenience, the terms probe array or microarray will generally be used broadly hereafter to refer to all of these types of arrays and parallel biological assays.

A microarray made by depositing or positioning pre-synthesized or pre-selected probes on a substrate, or by depositing/positioning techniques that may be developed in the future, may be referred to herein as a spotted array. Typically, but not necessarily, spotted arrays are commercially fabricated on microscope slides. These arrays often consist of liquid spots containing biological material of potentially varying compositions and concentrations. For instance, a spot in the array may include a few strands of short polymers, such as oligonucleotides in a water solution, or it may include a high concentration of long strands of polymers, such as complex proteins. The Affymetrix® 417™ and 427™ Arrayers are devices that deposit densely packed arrays of biological material on a microscope slide in accordance with these techniques. Aspects of these, and other, spot arrayers are described in U.S. Pat. Nos. 6,121,048, 6,040,193 and 6,136,269, in PCT Applications Nos. PCT/US99/00730 (International Publication Number WO99/36760) and PCT/US 01/04285, in U.S. patent applications Ser. Nos. 09/122,216, 09/501,099, and 09/862,177, and in U.S. Provisional Patent Application Ser. No. 60/288,403, all of which are hereby incorporated by reference in their entireties for all purposes. Other techniques for depositing or positioning biological probes on a substrate, i.e., creating spotted arrays, also exist. For example, U.S. Pat. No. 6,040,193 to Winkler, et al. is directed to processes for dispensing drops of biological material. The ″193 patent, and U.S. Pat. No. 5,885,837 to Winkler, also describe separating reactive regions of a substrate from each other by inert regions and spotting on the reactive regions. The ″193 and ″837 patents are hereby incorporated by reference in their entireties. Other techniques for producing spotted arrays are based on ejecting jets of biological material. Some implementations of the jetting technique use devices such as syringes or piezo electric pumps to propel the biological material.

Synthesized and spotted microarrays typically are used in conjunction with tagged biological samples such as cells, proteins, genes or EST″s, other DNA sequences, or other biological elements. These samples, sometimes referred to herein as targets, typically are processed so that they are spatially associated with certain probes in the probe array. In one non-limiting implementation, for example, one or more chemically tagged biological samples, i.e., the targets, are distributed over the probe array. Some targets hybridize with at least partially complementary probes and remain at the probe locations, while non-hybridized targets are washed away. These hybridized targets, with their tags or labels, are thus spatially associated with the targets″ complementary probes. The associated probe and target may sometimes be referred to as a probe-target pair. Detection of these pairs can serve a variety of purposes, such as to determine whether a target nucleic acid has a nucleotide sequence identical to or different from a specific reference sequence (see, for example, U.S. Pat. No. 5,837,832). Other uses include gene expression monitoring and evaluation (see, e.g., U.S. Pat. No. 5,800,992; U.S. Pat. No. 6,040,138; and International App. No. PCT/US98/15151, published as WO99/05323), genotyping (U.S. Pat. No. 5,856,092), or other detection of nucleic acids. The ″832, ″992, ″138, and ″092 patents, and publication WO99/05323, are hereby incorporated by reference herein in their entirety for all purposes.

To ensure proper interpretation of the term probe as used herein, it is noted that contradictory conventions exist in the relevant literature. The word probe is used in some contexts to refer not to the biological material that is synthesized or deposited on a substrate, as described above, but to what has been referred to herein as the target. To avoid confusion, the term probe is used herein to refer to the elements synthesized or deposited on a substrate to respectively create synthesized or spotted microarrays.

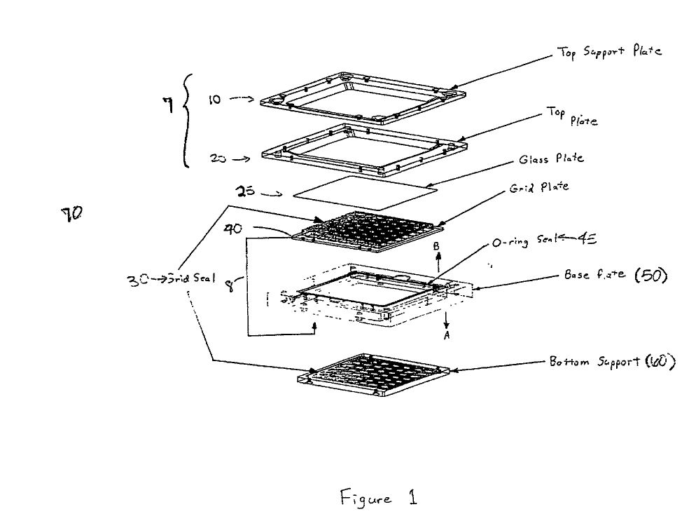

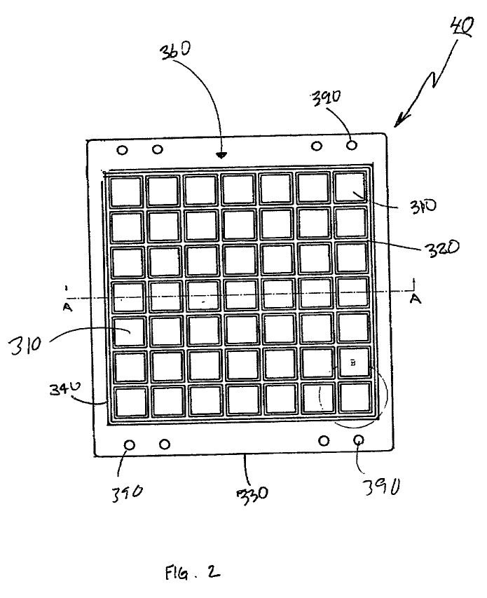

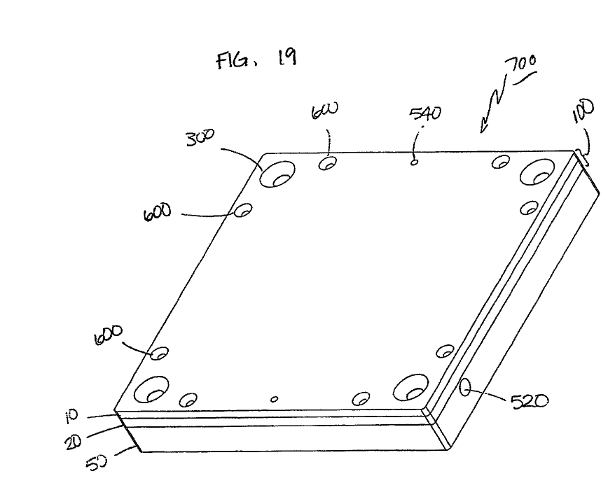

FIG. 1 is an exploded view of a particular embodiment of an apparatus for processing multiple microarrays, referred to as multiple-array processing apparatus 5. An assembled version of this embodiment is illustrated in FIGS. 19-21. In accordance with the illustrated implementation, a top segment 7 is composed of a top support plate 10 and a top plate 20. A base segment 8 includes a base plate 50 and a bottom support plate 60. As shown in FIGS. 2-4, grid plate 40 creates walls of multiple chambers 310, each one of which may be considered the walls of a sample area enclosing a microarray. In particular, walls 320 extend from the surface to form a pattern of through holes of grid plate 40. As noted, it is preferred that the walls 320 be relatively impermeable to any fluid that may be stored in chamber 310, so as to retain the fluid within the chambers 310 and avoid cross-contamination among chambers 310. In this embodiment, walls 320 extend generally perpendicularly to the surfaces 327, and thus may be referred to for convenience as vertically oriented. As noted, however, the apparatus in use may be disposed in any orientation, and thus terms such as vertical are used for illustrative purposes only.

Grid seal 30 also assists in the formation of a sealed chamber and in avoidance of cross contamination. It is positioned preferably on top surfaces 327 of the vertical walls 320 within a groove 380 formed in the top surfaces 327. Grid seal 30 preferably is formed from a compliant, flexible, inert material, for example silicone, although any suitable sealing material may be used. Any cross sectional profile may be selected for grid seal 370 so that a uniform seal can be achieved, preferably having a low contact pressure, for example a substantially tubular or u-shaped profile may be selected. The groove 380 in grid plate 40 is selected to retain grid seal 30 of the selected profile with friction contact, also promoting low insertion forces. Alternatively, an adhesive or other mechanical retaining force may be used to retain grid seal 30 in place. Preferably, the grid segment 40 is machined out of anodized aluminum, and coated with Teflon. However, it will be appreciated that grid plate 40 may be constructed out of other materials which provide a liquid tight seal that is resistant to bacterial infection or phage contamination.

In this embodiment, the grid segment 40 is constructed as a grid plate, and these terms are used interchangeably. However, it should be appreciated that other configurations that accommodate multiple arrays of plate-based samples may also be used. Similarly, the grid seal 30 may be molded out of other flexible, inert, and liquid tight materials.

Referring to FIG. 2, grid plate 40 creates an array 360 of individual sample chambers 310. While array 360 is depicted as a 7×7 array in FIG. 2, other sizes of arrays may be configured, such as 12×12, 16×16, 24×24, 32×32, 64×64, 96×96, 24×32, 12×24 and so on. Walls 320 separate each individual sample chamber 310 by creating four walls. Alternatively, individual sample chambers 310 may be composed of 3 or 5 or any number of sides using walls 320.

In the illustrated embodiment, an outer edge 340 of grid plate 40 contains mating groove 380, which positions grid seal 30. Fasteners 395 such as screws may be used in order to fasten and aligned this grid segment 40 with other segments comprising this apparatus, through apertures, such as the illustrated apertures 390. Any form of fastener may be used that provides sufficient strength for retaining the structural integrity of stacked array 5, such as for example, guide pins, rivets, nails, nut/bolt combinations, Velcro®, and so on. Preferably a releasable fastener is used, such as a screw or nut/bolt combination, although permanent type fasteners may be desired in some applications, such as adhesives. In the illustrated example fastener(s) 395 connect grid plate 40 in to a bottom support plate 60 via aperture(s) 390.

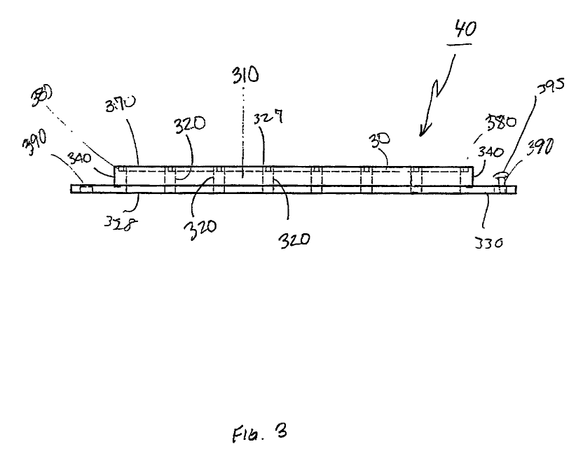

FIG. 3 illustrates a cross section of a preferred embodiment of grid segment 40. Apertures 390 are positioned at desired locations, and assist in aligning connecting segments, thus preserving the integrity of each individual sample chamber 310. Other aligning features may be used to assist in aligning segments. For example, mating grooves 380 are positioned on either end of grid plate 40 on the outer edge 340. The chambers 310 of array 360, including respective walls 320 extend from the are raised vertically in relation to frame 330. Atop each impermeable wall 320 rests grid seal 30, preferably with very low contact pressure. Preferably, grid seal 30 creates a liquid tight seal between the impermeable wall 320 and plate member 25. In this manner, each individual chamber 310 in array 360 is isolated chemically from each other individual sample chamber 310.

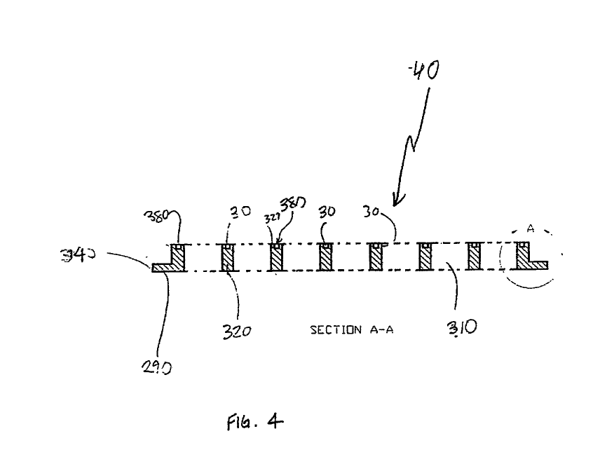

FIG. 4 illustrates mating groove 380 embedded in top surfaces 327 of each impermeable wall 320 allowing for positioning and placement of grid seal 30. It will be appreciated that groove 380 may be any other placement configuration that allows the grid seal 30 to seal each individual chamber 310 and the corresponding array 360 uniformly, preferably resulting in very low contact pressure between the impermeable wall 320 and grid seal 30.

Grid segment 40 in this embodiment is a grid plate and has a precision machined surface which mates to a precision machined face on base segment 50. In this example, base segment 50 is a base plate. The mounting face for grid plate 40 is on the A side of the base segment as shown in FIG. 1. The base segment is machined out of 6061 aluminum and hard coat anodized. It will be appreciated that other fasteners such as magnets, adhesives, or other connecting devices known in the art may be used to connect segments together.

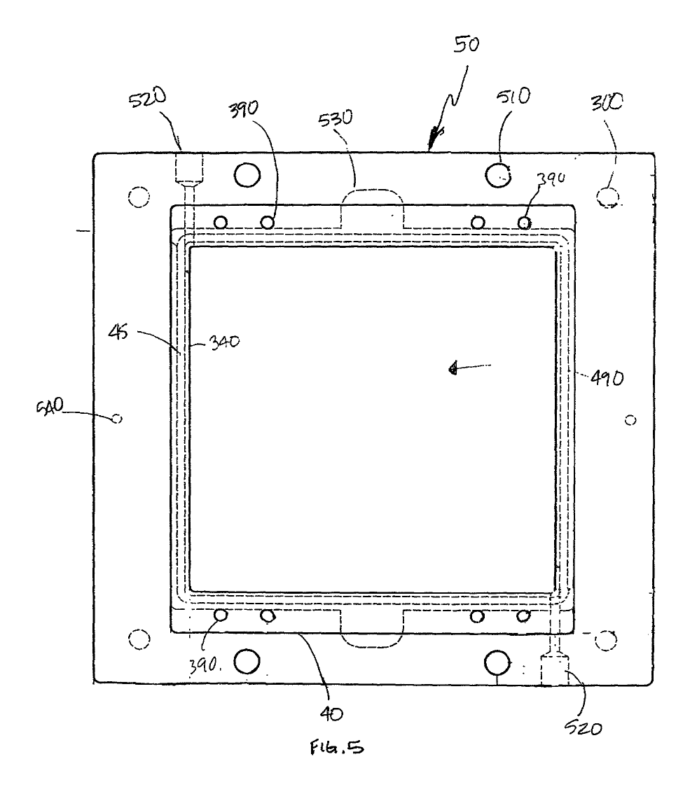

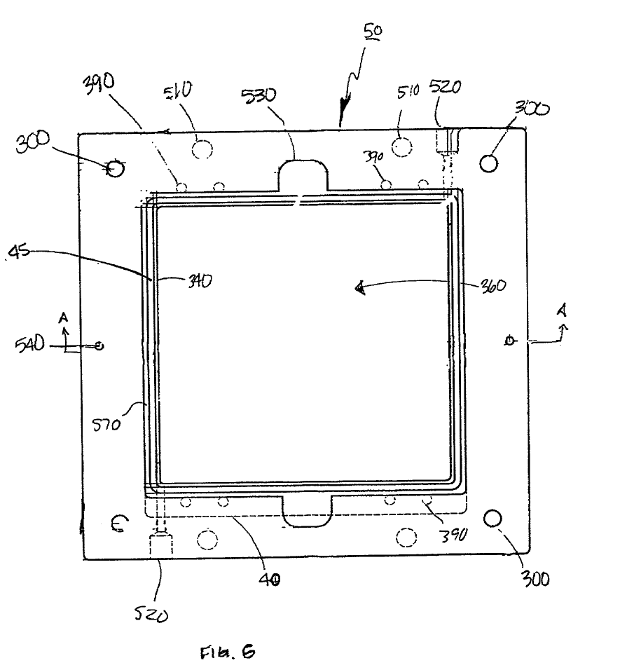

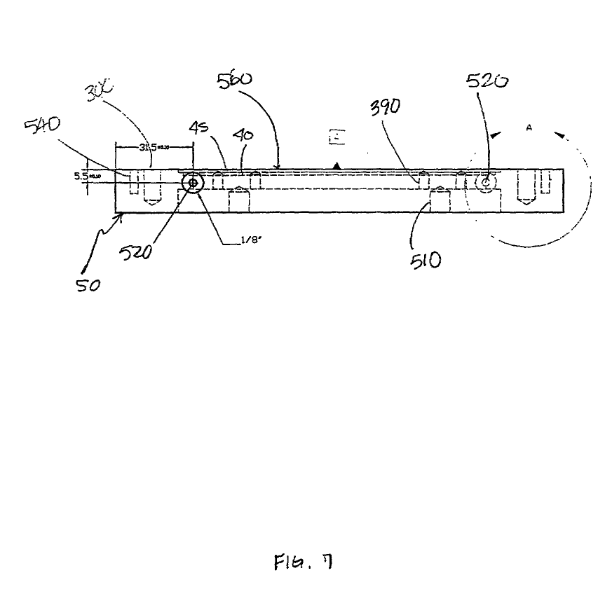

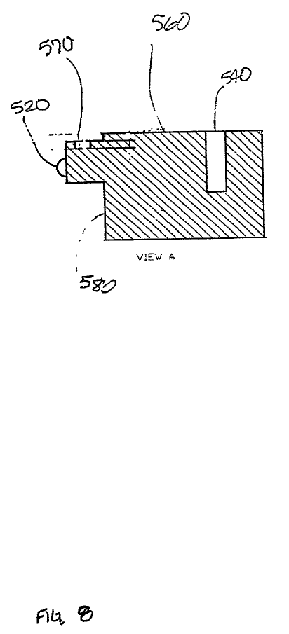

As assembled, the underside 328 of grid segment 40 is connected adjacent a base plate 50. FIGS. 5-8 illustrate the placement of grid plate 40 and base segment 50 together in assembled relation. As can be seen in these illustrations, apertures 510 serve to provide receive a fastener(s) to fasten the base plate 50 to top plate 20 and/or top support plate 10 and/or bottom support 60 via corresponding apertures in those elements. Any form of fastening arrangement may be used as described more fully above, although it is preferred that an aperture/mechanical fastener be used.

O-ring seal 45 is placed within base plate 50 preferably within receiving groove 570 although any arrangement to sufficiently mount the O-ring 45 on the base plate 50 and maintain a fluid-tight seal when assembled may be used. In assembled orientation, grid plate 40 is positioned on the side of seal 45 opposite the groove 570 and base plate 50. O-ring seal 45 is positioned in groove 570 in order to position and retain O-ring seal 45 with friction. At this point, O-ring seal 45 and grid seal 30 are facing the B side of base plate 50, as shown in FIG. 1. Pipe fitting 520 serve to introduce liquids and gasses into array 360 as desired. While this embodiment shows two pipe fittings 520, more or fewer pipe fittings may be used as desired. Apertures 540 may be used to employ removable guide pins to align a top segment 100 or any other segment with a bottom segment, such as the base plate 50. Apertures 300 are positioned in the four corners of base plate 50 and serve to connect base plate 50 with a top segment 100.

A plate member 25 is place in top of grid seal 30, forming an enclosure with the walls 320 and bottom support 60. Preferably plate member 25 is a transparent material, such as glass, although any material that can form an enclosure with grid seal 30 can be used. For example a plastic can be used as well. The plate member 25 preferably has microarrays positioned on discrete locations on its surface. These microarrays preferably are aligned with grid seal 30 and walls 320 such that the seal 30 does not contact any of the microarrays on glass plate 25 and preferably fluidically separates them from one another when the assembly 700 of the present invention is fully assembled. Thus sample chambers 310 are formed with plate member 25 as one surface of individual sample chambers 310 and with walls 320 and bottom support 60 forming the other sides of the chambers 310.

Referring now to FIGS. 9-11, top plate 20 contains apertures 300 that preferably align with corresponding apertures in one or more of the other segments of assembly 5. For example apertures 300 optionally may align with apertures 520 on base plate 50 and are used for positioning removable fasteners, preferably guide pins, that assist in alignment between top segment 100 and base plate 50. Apertures 600 are positioned on each side of top plate 20 and fasteners members connect top plate 20 to top support plate 10 to form top segment 100. Wedge 610 forms a rectangular frame surrounding the glass plate 25. Any form of fastener may be used, such as already discussed above.

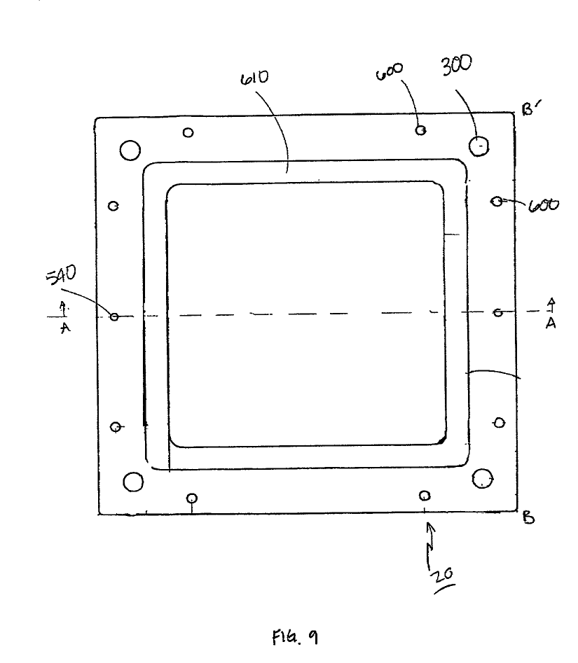

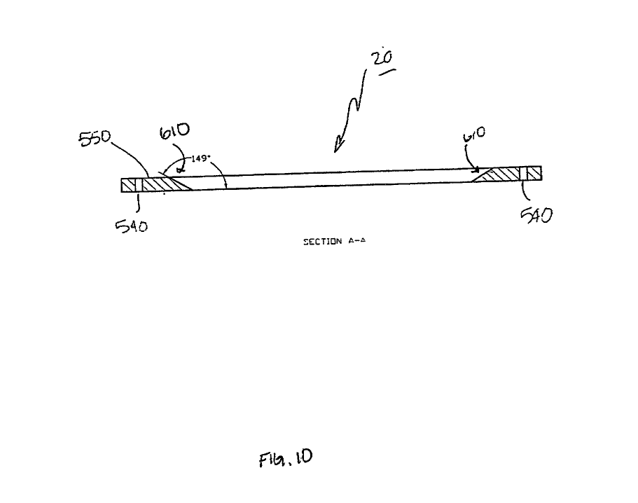

In the embodiment shown in FIGS. 10 and 11, wedge 610 forms an angle with the bottom surface of top plate 10. In the illustrated embodiment an angle of 149 degrees is provided although it readily will be appreciated that other angles may be selected as well. FIG. 10 shows a cross section of one embodiment of top plate 10 from FIG. 9. In this illustration, guide pins placed in apertures 540 are placed on opposite sides of top plate 10. Region 550 is an open area in which top support plate 10 fits. FIG. 11 is a side view of edge B as shown in FIG. 9, and illustrates apertures 300 and 600, wedge 610, apertures 600 on another edge of top plate 20, removable guide pin 540, and symmetrically matched elements 600, 610, 300, and 600 approaching edge B.

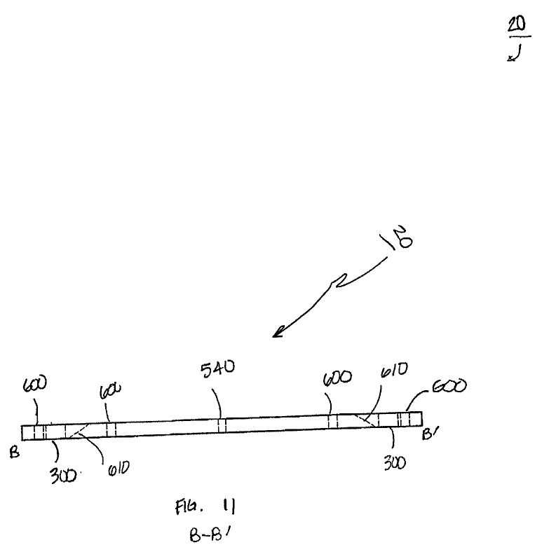

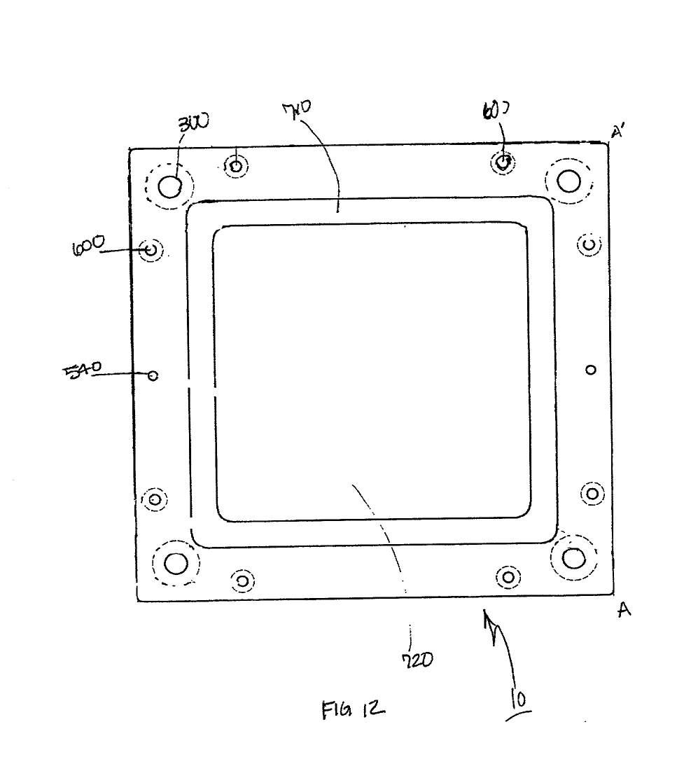

FIGS. 12 and 13 show one embodiment of top support plate 10. Top support plate 10 fits adjacent to top plate 20 and is fastened via fastening members (such as already described above) connected through apertures 300 and 600 located in top plate 20 and top support plate 10. Wedge 710, forms a similar rectangular frame to top plate 20 and wedge 710 fits snugly inside of wedge 610. Of course it is preferred wedges 710 and 610 be at similar or identical angles so as to mate snugly. Similarly, removable guide pin apertures 540 also align with their corresponding elements in FIGS. 9-11 on top plate 20. Once top plate 20 and top support plate 10 are aligned, wedge 710 fits snugly against wedge 610 providing a resulting bottom face 720 such that a continuous, stepless, smooth surface exists across the entire top segment with minimal gap between top plate 20 and top support plate 10. Removable guide pins 540 and apertures 300 and 600 assist in aligning the two plates 10 and 20 comprising top segment 100. It will be appreciated that apertures 300, 600, and 540 may be configured in any fashion and shape known in the art such that their respective segments are aligned and fastened. For example, the type of connecting members, such as a magnet, may dictate the shape, design, or position of elements 300, 600, or 540.

As assembled, top segment 100 is aligned with the base plate, with plate member 25 therebetween. Removable guide pins 540 that may be inserted from top segment 100 through like apertures on base plate 50 and facilitate alignment and to retain the stack together. In the assembly process, once top segment 100 rests against plate member 25, fastening members such as screws may be inserted into apertures 300 through base plate 50 such that the bottom surface 720 of top segment 100 is secured against top mounting face 560 of base plate 50, thereby creating a tight seal through contact of bottom surface 720 and face 560. The features of base plate 50, grid plate 40, O-ring seal 45, and grid seal 30 are designed such that grid seal 30 and O-ring 45 are aligned when metal-to-metal contact is achieved between top segment 100, base plate 50, and grid plate 40.

Once base plate 50 is attached to top segment 100, the entire assembly 70 as shown in FIGS. 1 and 19 can now be flipped over if desired such that the A side is face up. Test sample may now be introduced into one or more of the now formed sample chambers 310. Samples may be introduced singly, serially, in parallel, or by any other means known in the art. Likewise, samples may be introduced manually or robotically. Once sealed, the individual test chambers 310 preferably are chemically isolated from one another, minimizing or eliminating cross contamination between chambers 310. By forming such discrete chambers, multiple tests can be performed in a single assembly 5. It is thus a feature of the invention that a microarray processing systems created in a segmented processing array assembly.

In operation, once samples have been introduced into chambers 310 such as via placing them on plate member 25, the array 360 of sample chambers 310 may be sealed simultaneously by fastening bottom support 60 to base plate 50 such as for example via apertures 300 and attendant fastening devices 395.

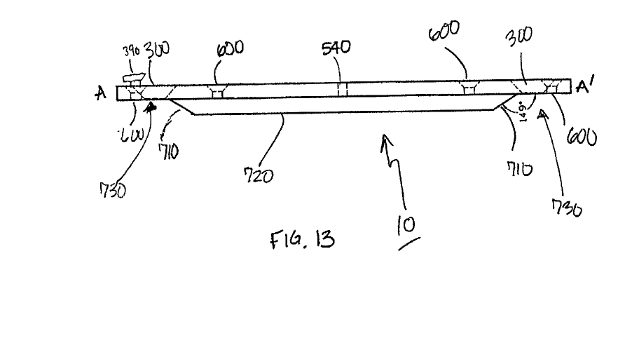

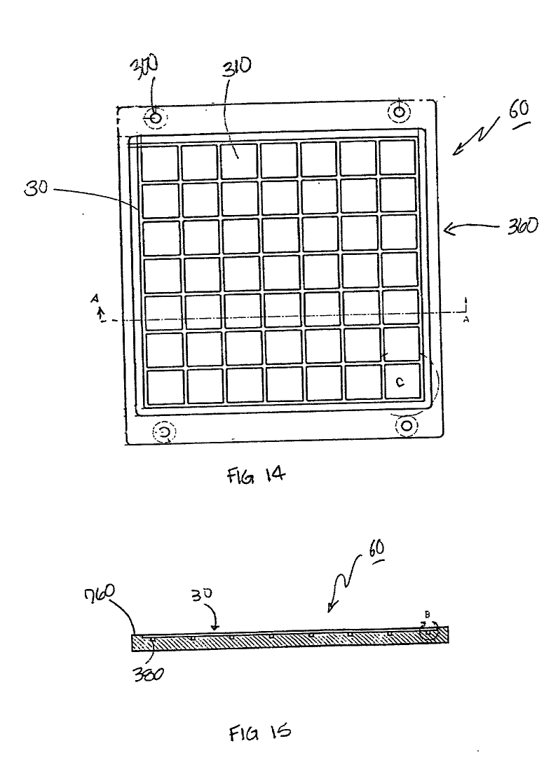

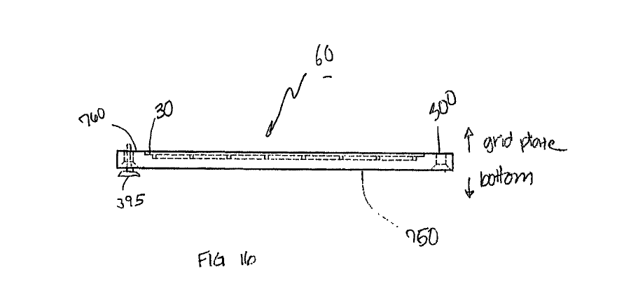

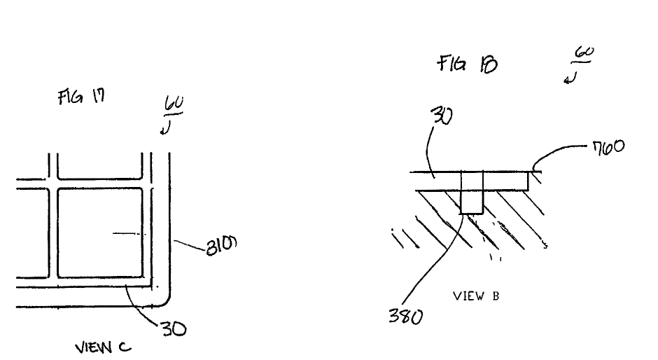

FIGS. 14-18 illustrate an embodiment of a bottom support 60. In order to make each individual sample chamber 310 fluid-tight, a grid seal 30 preferably is installed into bottom support 60 as shown in FIGS. 14-18. Exemplary structures for grid seals and mounting arrangements for grid seals already have been discussed above, and apply equally well to the sealing arrangement used in bottom support 60. Grid seal 30 is positioned in mating grooves 380, preferably providing a seal with relatively low contact pressure. Bottom support 60, including grid seal 30, is aligned using apertures 300 or other alignment members such that grid seal 30 will effectively seal each individual sample chamber 310 in the array of sample chambers 360 with surface 750 creating a cover for chambers 310, thereby preventing contamination and evaporation. A metal-to-metal contact is achieved between the top surface 760 of bottom support 60 and the bottom surface 290 of grid plate 40. Once sealed, the entire assembly can now be transported, tested, or otherwise processed.

FIGS. 19-21 show the assembled assembly 700. As can be seen in FIG. 19, top support plate 10 top plate 20, comprising top segment 100, and base plate 50 are aligned using apertures 300 and removable guide pins 540 or other fasteners. Fasteners also may be connected through apertures 600 to connect and align top support plate 10 with top plate 20 to form top segment 100. Metal-to metal contact is achieved between the surfaces of top support plate 10 and top plate 20, as well as between the surfaces between top plate 20 and base plate 50, in this implementation.

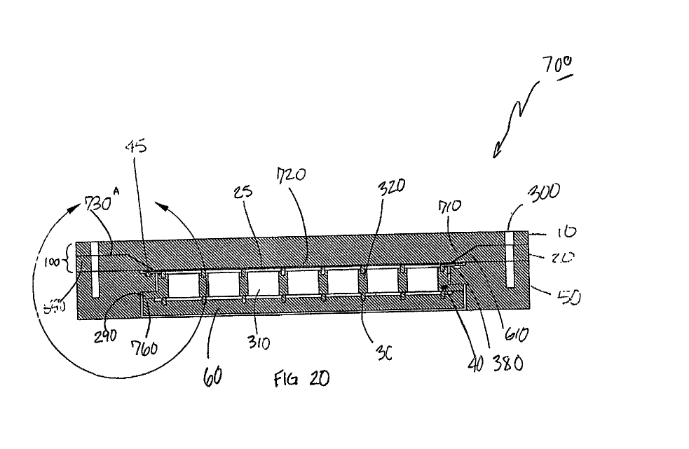

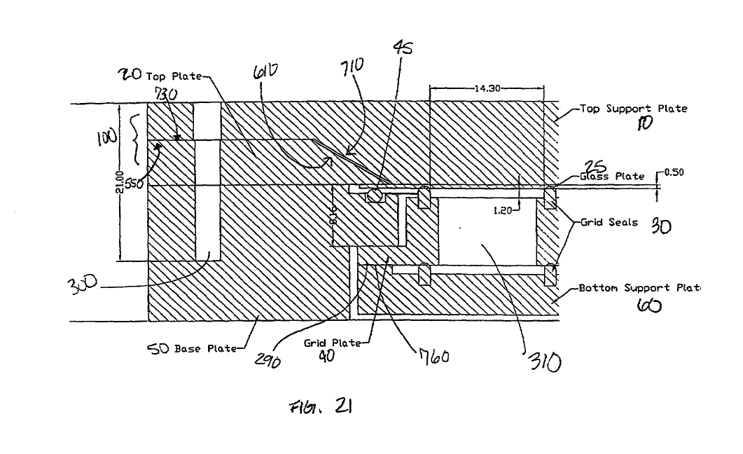

FIG. 20 shows a cross section of assembled apparatus 700. From this view, one can see the bottom mounting surface 730 of top support plate 10 is wedged against the top surface 550 of top plate 20 wherein wedges 710 and 610 also preferably fit snugly. Bottom surface 720 of top support plate 10 directly contacts plate member 25. Plate member 25 contacts grid seal 30 which is embedded in grooves 380 in grid plate 40. Grid seal 30 rests directly on top of walls 320, such as on surface 327. Walls 320 form the walls of individual sample chambers 310 of which plate member 25 forms one surface as well. Grid seal 30 embedded in bottom support plate 60 rest atop of grid plate 40 and forms another surface of chambers 310. FIG. 21 shows a detailed view corresponding to FIG. 20.

In one embodiment, the samples may be processed to prepare the samples for detecting, typically an imaging process. These pre-detection processes usually include removing the samples and then flowing liquids or gases across the samples to wash, stabilize, stain, or otherwise prepare the sample. In a preferred embodiment, bottom support 60 may be removed from the assembly 700 and the samples within each individual sample chamber 310 removed and recovered via a singular or multiple tip pipettor. Another liquid can be introduced into each individual chamber 310 if desired, in order to prevent chamber 310 from drying out before further processes, such as flow processes, are started. For example, the following flow cell processes are preferably administered collectively to the array of sample chambers 360 in order to minimize variation and simplify processes. Optionally, however these processes may be performed individually on each individual sample chamber 310.

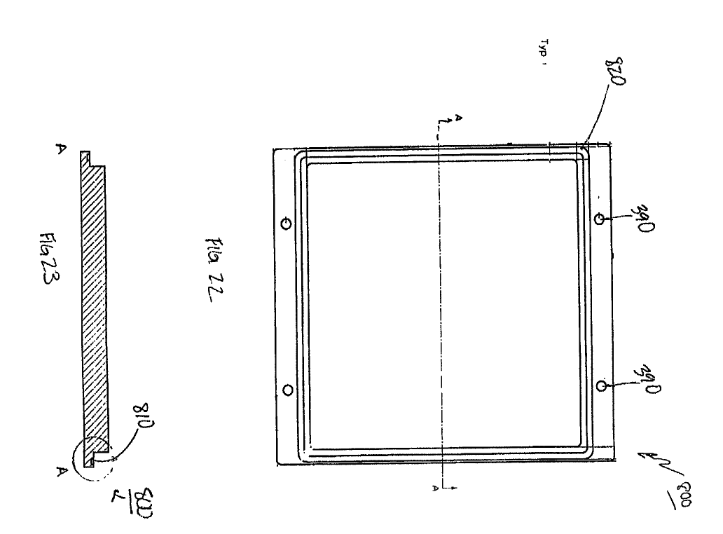

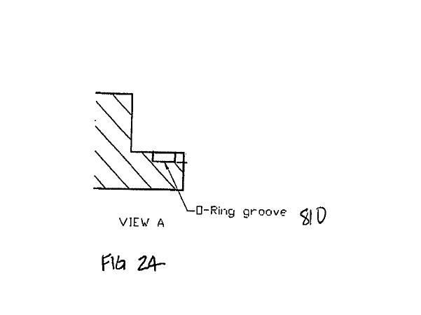

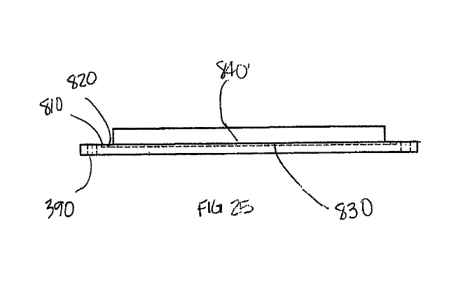

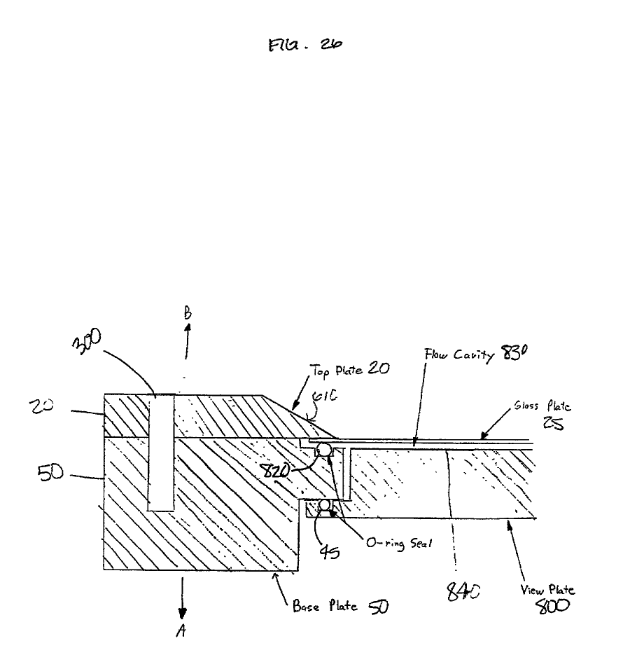

A single flow cell chamber or cavity 830 may be created by replacing grid plate 40 from base plate 50 with view plate 800, as shown in FIGS. 22-26. In view plate 800, O-ring seal 820 is positioned in O-ring groove 810 such that a low volume, sealed chamber is created across the array of sample chambers 360. Apertures 390 fasten and align view plate 800 with like apertures in base plate 50. In one embodiment, base plate 50 contains pipe fittings 520 such that when view plate 800 is installed, liquids or gases may be passed over all sample chambers in array 360 uniformly and quiescently. Flow cell chamber 830 may be optionally filled with liquid or gas and then sealed with valves on base plate pipe fittings 520. In a preferred embodiment, liquids or gases may be introduced via fittings 520 while apparatus is positioned directly on a detecting device, thus offering a controlled environment directly within the each sample chamber 310 during detection.

In another preferred embodiment, top support plate 10 may also be removed such that plate member 25 is visible from side B as seen in FIG. 26. FIG. 26 shows one embodiment in which view plate 800 has replaced grid plate 40 in base plate 50. As can be seen, top plate 20 is aligned with base plate 50 via apertures 300, fastened with screws. O-ring seal 45 helps retain the positioning of view plate 800 adjacent to base plate 50. O-ring 820 creates flow chamber 830 between glass plate 25 and the top surface 840 of view plate 800. Once top support plate 10 is removed, detection processes such as spectrophotometry or other optical methods may be initiated through glass plate 25. Practitioners in the art will understand that other detection methods known in the art may be used in accordance with the present invention.

In one specific experimental implementation of the preceding embodiment, the assembly is a stacked, integrated device that can accommodate a 12.5 cm×12.5 cm glass sample segment containing 49 oligonucleotide (e.g., GeneChip®-type) microarrays arranged as a 7×7 array of microarrays. The device serves as a hybridization chamber for 49 different samples as well as the flow-cell during subsequent processing such as washing and staining. In this implementation, the assembly includes a two-piece frame, such as a top segment and a bottom segment holding the sample segment in place. Different modules can be attached on either side of the sample segment at different processing stages. During hybridization, the arrays and samples are kept separated from each other by a grid seal, such as a silicone seal held in place and pressed against the sample segment by a coated aluminum grid segment. A base segment, incorporating a bottom support such as solid aluminum plate attached to the frame provides support from the back to prevent breaking of the glass sample segment. Hybridization samples are applied to the arrays from above through the open grid. A volume of 300 μL is sufficient to completely cover each array in this implementation. Evaporation is prevented by a solid lid pressing a second seal onto the grid plate (not shown). Following hybridization, the samples are recovered for possible reuse and wash buffer is added onto the arrays to prevent them from drying out.

In this implementation, to convert the hybridization chamber into a flow-cell for washing and staining of all the arrays in parallel, the grid plate is removed and a solid coated aluminum plate, held at a distance of 1.5 from the sample segment, is attached in its place. This creates a space between the oligonucleotides attached to the sample segment and the solid plate that can be filled and vented through two ports (inlet and outlet ports) in the frame. Removing the back support plate allows viewing of the sample segment and completes the conversion into a flow-cell with a total volume of about 35 ml. Two sets of washes of increasing stringency are performed after the hybridization to remove sample RNA non-specifically bound to the arrays. As in the standard procedure for Affymetrix® GeneChip® oligonucleotide microarrays, the entire sample segment is then stained with streptavidin-conjugated phycoerythrin, followed by further signal amplification with biotinylated anti-streptavidin and a second staining with streptavidin-conjugated phycoerythrin.

The described experimental approach greatly increases the rate at which, for example, gene expression profiles can be generated using microarrays and, continuing this example, facilitates the construction of large databases of gene expression patterns. Typically, both the sample preparation and whole sample segment hybridization may be accomplished by one person in little more time and effort than required to process a few individual samples and microarrays. Furthermore, many steps may be readily automatable and multiple plates and sample segments may be processed in parallel. Moreover, while sample segments with 7×7 individual microarrays were described with respect to this experimental implementation, sample segments with larger numbers of microarrays could be used. Also, as noted, the described apparatus and methods are generalizable and, as will now be appreciated by those of ordinary skill in the art in view of this disclosure, may be applied, for example, to cDNA arrays, SNP arrays, sequencing arrays, and tag arrays.

Having described various embodiments and implementations of the present invention, it should be apparent to those skilled in the relevant art that the foregoing is illustrative only and not limiting, having been presented by way of example only. For example, many other schemes for distributing functions among the various elements of the illustrated embodiment are possible in accordance with the present invention. The functions of any element may be carried out in various ways in alternative embodiments. Also, the functions of several elements may, in alternative embodiments, be carried out by fewer, or a single, element. Numerous other embodiments, and modifications thereof, are contemplated as falling within the scope of the present invention as defined by appended claims and equivalents thereto.

What is claimed is:

1. A method for analyzing nucleic acids using a plurality of nucleic acid microarrays, comprising the steps of:
(1) preparing a cell sample having nucleic acids; and
(2) contacting the sample with an apparatus that comprises one or more containing members constructed and arranged to contain the plurality of nucleic acid microarrays, and
a separating member constructed and arranged so that, when the separating member is disposed in a first position with respect to the containing members, at least two of the plurality of nucleic acid microarrays are fluidically separated from each other by the separating member, and when the separating member is disposed in a second position with respect to the containing members, the at least two microarrays are fluidically coupled with each other.

2. An apparatus for processing a plurality of microarrays disposed on a substrate, comprising:
one or more containing members constructed and arranged to contain the substrate; and
a separating member constructed and arranged so that, when the separating member is disposed in a first position with respect to the containing members, at least two of the plurality of microarrays are fluidically separated from each other by the separating member, and
when the separating member is disposed in a second position with respect to the containing members, the at least two microarrays are fluidically coupled with each other.

3. The apparatus of claim 2, wherein:
the one or more containing members include a first segment and a second segment in contact with the first segment, wherein the substrate is disposed between the first and second segments.

4. The apparatus of claim 3, wherein:
the separating member is disposed between the first and second segments when the separating member is in the first position, and is disposed apart from the first and second segments when the separating member is in the second position.

5. The apparatus of claim 3, wherein:
the substrate is retained in place by the first and second segments.

6. The apparatus of claim 3, wherein:
the first segment includes a central frame.

7. The apparatus of claim 6, wherein:
the central frame includes an inlet port for receiving fluids and an outlet port for expelling fluids.

8. The apparatus of claim 2, wherein:
the separating member includes one or more walls constructed and arranged to fluidically separate the at least two microarrays when the separating member is disposed in the first position.

9. The apparatus of claim 8, wherein:
the separating member includes a grid plate.

10. The apparatus of claim 9, wherein:
the grid plate includes a plurality of grid elements determined by the one or more walls, wherein each of the at least two microarrays is fluidically separated from each of the other at least two microarrays by a grid element when the separating member is disposed in the first position, and wherein each of the at least two microarrays is fluidically coupled with the other at least two microarrays when the separating member is disposed in the second position.

11. The apparatus of claim 10, wherein:
the plurality of grid elements is equal in number to the plurality of microarrays.

12. The apparatus of claim 2, wherein:
the plurality of microarrays include synthesized probe arrays wherein the probes comprise oligonucleotides.

13. The apparatus of claim 12, wherein:
the oligonucleotides are synthesized to the microarrays based, at least in part, on photolithography.

14. The apparatus of claim 2, wherein:
the plurality of microarrays are disposed on a contiguous surface of the substrate.

15. The apparatus of claim 14, wherein:
the contiguous surface of the substrate comprises a photolithographic wafer.

16. An apparatus for processing a plurality of microarrays disposed on a substrate, comprising:
one or more containing members including a first segment and a second segment, wherein the substrate is disposed between the first and second segments; and
a separating member including a grid plate having a plurality of grid elements constructed and arranged so that,
when the separating member is disposed in a first position with respect to the containing members, at least two of the plurality of microarrays are fluidically separated from each other by one or more of the grid elements, and
when the separating member is disposed in a second position with respect to the containing members, the at least two microarrays are fluidically coupled with each other.

17. A method for processing a plurality of microarrays, comprising the steps of:
(1) providing a substrate upon which the microarrays are disposed;
(2) fluidically separating at least two of the plurality of microarrays from each other;
(3) contacting the at least two microarrays with one or more target solutions while the at least two microarrays are fluidically separated;
(4) retaining the fluidic separation of the at least two microarrays for a first period of time sufficient for hybridization reactions, if any, to occur between the target solutions and the at least two microarrays;
(5) fluidically coupling the at least two microarrays after the first period has elapsed; and
(6) performing one or more parallel fluidic processes on the at least two microarrays based, at least in part, on the fluidic coupling.

18. The method of claim 17, further comprising the step of:
(7) removing at least a portion of the one or more target solutions after the first period has elapsed and prior to performing step (5).

19. The method of claim 17, wherein:
the one or more fluidic processes include one or more of the group consisting of removing at least a portion of the one or more target solutions, washing, staining, or preserving.

20. The method of claim 17, further comprising the step of:
(7) providing one or more containing members including a first segment and a second segment in contact with the first segment, wherein the substrate is disposed between the first and second segments.

21. The method of claim 17, wherein:
the substrate comprises a contiguous surface.

22. The method of claim 17, wherein:
the microarrays include synthesized probe arrays.

23. The method of claim 17, wherein:
step (2) includes disposing a grid plate having a plurality of grid elements on the substrate in a first position so that each of the at least two microarrays is aligned with a grid element.

24. The method of claim 23, wherein:
the grid elements include walls that, when the grid plate is in the first position, contribute to fluidically separating the at least two microarrays from each other.

25. The method of claim 23, wherein:
step (5) includes moving the grid plate in the first position to a second position away from the substrate.

26. The method of claim 17, wherein:
the plurality of microarrays include synthesized probe arrays wherein the probes comprise oligonucleotides.

27. A method for processing a plurality of microarrays, comprising the steps of: (1) providing one or more containing members including a first segment and a second segment in contact with the first segment;
(2) disposing a substrate between the first and second segments, wherein the plurality of microarrays are disposed on a contiguous surface of the substrate comprising a photolithographic wafer;
(3) fluidically separating at least two of the plurality of microarrays from each other;
(4) contacting the at least two microarrays with one or more target solutions while the at least two microarrays are fluidically separated;
(5) retaining the fluidic separation of the at least two microarrays for a first period of time sufficient for hybridization reactions, if any, to occur between the target solutions and the at least two microarrays;
(6) fluidically coupling the at least two microarrays after the first period has elapsed; and
(7) performing one or more parallel fluidic processes on the at least two microarrays based, at least in part, on the fluidic coupling.

28. A microarray processing system, comprising:
a first segment;
a second segment in contact with the first segment; and
a processing array positioned between the first segment and the second segment, and retained in place by the first and second segments.

29. The microarray processing system of claim 28, wherein: the processing array includes
a plate member between the first and second segment, wherein the plate member includes a first surface,
a grid segment containing an array of chamber walls, and
a bottom support segment,
wherein, when the grid segment is disposed in a first position between the bottom support segment and the plate member, multiple processing chambers are formed that each include as a first chamber surface a portion of the first surface of the plate member, as a second chamber surface opposed to the first chamber surface a portion of the bottom support segment, and as walls an array element of the array of chamber walls.

30. The microarray processing system of claim 29, further comprising:
a first grid seal between the plate member and the grid segment, and
a second grid seal between the grid segment and the bottom support segment.

31. The microarray processing system of claim 29, wherein:
a plurality of microarrays are disposed on the first surface of the plate member, and
when the grid segment is disposed in the first position, the multiple processing chambers align with and fluidically separate the plurality of microarrays.

32. The microarray processing system of claim 31 wherein:
the grid segment is movable between the first position and a second position in which the multiple processing chambers are not aligned with the plurality of microarrays.

33. The microarray processing system of claim 31, wherein:
the grid segment is movable between the first position and a second position in which the multiple processing chambers do not fluidically separate the plurality of microarrays.
